(19) United States
(12) Patent Application Publication
(10) Pub. No.: US 2023/0241296 A1
(43) Pub. Date: Aug. 3, 2023

(54) POSITIVE DISPLACEMENT PUMPING MECHANISM WITH DOUBLE RESERVOIR

(71) Applicant: INSULET CORPORATION, Acton, MA (US)

(72) Inventors: Soroush KAMRAVA, Everett, MA (US); Steven CARDINALI, Tewksbury, MA (US); Daniel ALLIS, Boxford, MA (US)

(21) Appl. No.: 18/160,409

(22) Filed: Jan. 27, 2023

Related U.S. Application Data
(60) Provisional application No. 63/304,270, filed on Jan. 28, 2022.

(51) Int. Cl. A61M 1/00

(52) U.S. Cl. CPC A61M 1/00; A61F 2007/0059

(57) ABSTRACT

Disclosed herein is a double reservoir configuration for a spatially efficient pumping mechanism comprising an outer reservoir and an inner reservoir, wherein a linear translation of the inner reservoir causes the inner reservoir to move into the outer reservoir and act as a plunger for the outer reservoir to force fluid contained in the outer reservoir through a first fluid port. A static plunger disposed within the inner reservoir causes fluid disposed within the inner reservoir to be forced through a second fluid port. Also disclosed are various drive mechanisms for causing the linear translation of the first reservoir into the second reservoir.

### RELATED APPLICATIONS

This application claims the benefit of U.S. Provisional Pat. Application No. 63/304,270, filed Jan. 28, 2022, the contents of which are incorporated herein by reference in their entirety.

### BACKGROUND

Many conventional automatic drug delivery systems are well known, including, for example, wearable drug delivery devices of the type shown in FIG. 2. The drug delivery device 102 can be designed to deliver any type of liquid drug to a user. In specific embodiments, the drug delivery device 102 can be, for example, an OmniPod® drug delivery device manufactured by Insulet Corporation of Acton, Massachusetts. The drug delivery device 102 can be a drug delivery device such as those described in U.S. Pat. No. 7,303,549, U.S. Pat. No. 7,137,964, or U.S. Pat. No. 6,740,059, each of which is incorporated herein by reference in its entirety.

Drug delivery device 102 typically includes a positive displacement pump mechanism. Typically, the pump mechanism comprises a reservoir that stores the liquid drug. The liquid drug stored in the reservoir may be delivered to the user by expelling the drug from a reservoir using a driven plunger that longitudinally translates through the reservoir to force the liquid drug through a fluid port defined in the reservoir. The plunger may be longitudinally translated through the reservoir by, for example, a rigid leadscrew which pushes the plunger forward during pumping. When the reservoir is filled, the leadscrew travels backwards with the plunger. The leadscrew extends past the back of the plunger a distance equal to the stroke of the plunger plus an additional amount to allow for engagement with the drive mechanism. This leads to a space efficiency constraint when scaling the design. If the stroke of the plunger increases, the length of the leadscrew must increase by the same amount.

In wearable, on-body devices, it is desirable to keep the pump mechanism, as well as the overall drug delivery device 102, as small as possible to minimize the impact to the wearer. Additionally, because such drug delivery devices are typically powered by an on-board battery, it is desirable to minimize the power required to operate the device.

Therefore, it would be desirable to replace the prior art pump mechanism with a positive displacement pump having a more space-efficient pumping mechanism to allow for a smaller device, which would decrease the burden on the user.

### DEFINITIONS

As used herein, the term “liquid drug” should be interpreted to include any drug in liquid form capable of being administered by a drug delivery device via a subcutaneous cannula, including, for example, insulin, GLP-1, pramlintide, morphine, blood pressure medicines, chemotherapy drugs, fertility drugs, or the like, or co-formulations of two or more of GLP-1, pramlintide, and insulin.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A primary embodiment of the invention disclosed herein uses a double reservoir configuration having a larger, outer reservoir and a smaller, inner reservoir wherein the inner reservoir has a cross-sectional shape slightly smaller than the outer reservoir such that the inner reservoir can linearly translate through the outer reservoir, acting as a plunger for the outer reservoir. The two reservoirs are in fluid communication with each other via a rigid hollow rod which is disposed between the inner and outer reservoirs and which supports a static plunger for the inner reservoir such that, as the inner reservoir is linearly translated into the outer reservoir, the inner reservoir forces a fluid from the outer reservoir, through the hollow rod and into the inner reservoir. The static plunger in the inner reservoir acts to force fluid from the inner reservoir through an outlet fluid port as the inner reservoir is linearly translated into the outer reservoir.

The double reservoir configuration takes advantage of the second reservoir and static plunger to use the space taken by the leadscrew in prior art examples of the drug delivery device. This makes embodiments using the double reservoir configuration more space efficient than prior art examples. Variations of the primary embodiment are directed to various ways of driving the inner reservoir into the outer reservoir and are discussed in detail below.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a functional block diagram of an exemplary system suitable for implementing the systems and methods disclosed herein.

FIG. 2 is a depiction of a prior art wearable drug delivery device of the type in which the invention disclosed herein would be used.

FIGS. 3 (A-D) are schematic, cross-sectional views of a primary embodiment of the invention, showing the outer reservoir, the inner reservoir, the hollow connecting rod and static plunger, as the device is being filled with a fluid and as the fluid is being dispensed.

FIGS. 4 (A-B) are opaque, perspective views of a first variation of the primary embodiment in which a leadscrew coupled to the inner reservoir is rotated by a tube nut to drive the inner reservoir into the outer reservoir.

FIGS. 5 (A-C) are opaque, perspective views of a second variation of the primary embodiment in which a leadscrew is in threaded engagement with a pusher element which pushes on a rear surface of the inner reservoir to drive the inner reservoir into the outer reservoir as the leadscrew rotates.

FIGS. 6 (A-B) are opaque, perspective views of a third variation of the primary embodiment in which a leadscrew is in threaded engagement with the inner reservoir such that rotation of the leadscrew drives the inner reservoir into the outer reservoir.

FIGS. 7 (A-B) are opaque, perspective views of fourth variation of the primary embodiment in which a rack and pinion configuration is used to move the outer reservoir toward the inner reservoir so as to drive the inner reservoir into the outer reservoir.

FIGS. 8 (A-B) are opaque, perspective views of a fifth variation of the primary embodiment in which two leadscrews are in threaded engagement with the inner reservoir such that a synchronized rotation of the leadscrews drives the inner reservoir into the outer reservoir.

FIG. 9 is a schematic, cross-sectional view of a secondary embodiment in which the inner reservoir is driven into the outer reservoir using a central leadscrew and which requires two fluid ports.

FIGS. 10 (A-B) are opaque, cutaway views of the secondary embodiment of the invention in which a central leadscrew within the inner reservoir is used to drive the inner reservoir into the outer reservoir.

FIGS. 11 (A-C) show different exemplary embodiment of reservoirs having different cross-sectional shapes.

FIGS. 12 (A-B) show two possible embodiments of a configuration for plunger suitable for use in all embodiments of the present invention.

### DETAILED DESCRIPTION

This disclosure presents various systems, components and methods for moving a liquid drug from a liquid reservoir in a drug delivery device to a patient interface, such as a needle or cannula. The embodiments described herein provide one or more advantages over conventional, prior art systems, components and methods, namely, a smaller overall footprint of the drug delivery device.

Various embodiments of the present invention include systems and methods for delivering a medication to a user using a drug delivery device (sometimes referred to herein as a “pod”), either autonomously, or in accordance with a wireless signal received from an electronic device. In various embodiments, the electronic device may be a user device comprising a smartphone, a smart watch, a smart necklace, a module attached to the drug delivery device, or any other type or sort of electronic device that may be carried by the user or worn on the body of the user and that executes an algorithm that computes the times and dosages of delivery of the medication.

For example, the user device may execute an “artificial-pancreas” (AP) algorithm that computes the times and dosages of delivery of insulin. The user device may also be in communication with a sensor, such as a glucose sensor or a continuous glucose monitor (CGM), that collects data on a physical attribute or condition of the user, such as a glucose level. The sensor may be disposed in or on the body of the user and may be part of the drug delivery device or may be a separate device.

Alternatively, the drug delivery device may be in communication with the sensor in lieu of or in addition to the communication between the sensor and the user device. The communication may be direct (if, e.g., the sensor is integrated with or otherwise a part of the drug delivery device) or remote/wireless (if, e.g., the sensor is disposed in a different housing than the drug delivery device). In these embodiments, the drug delivery device contains computing hardware (e.g., a processor, memory, firmware, etc.) that executes some or all of the algorithm that computes the times and dosages of delivery of the medication.

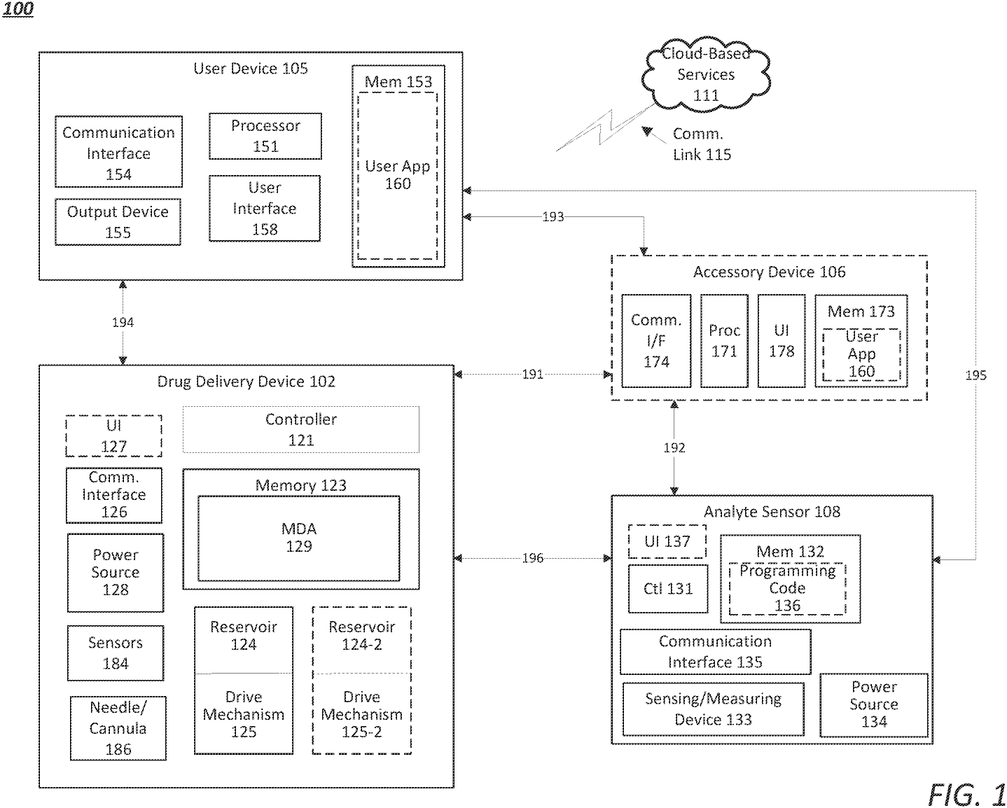

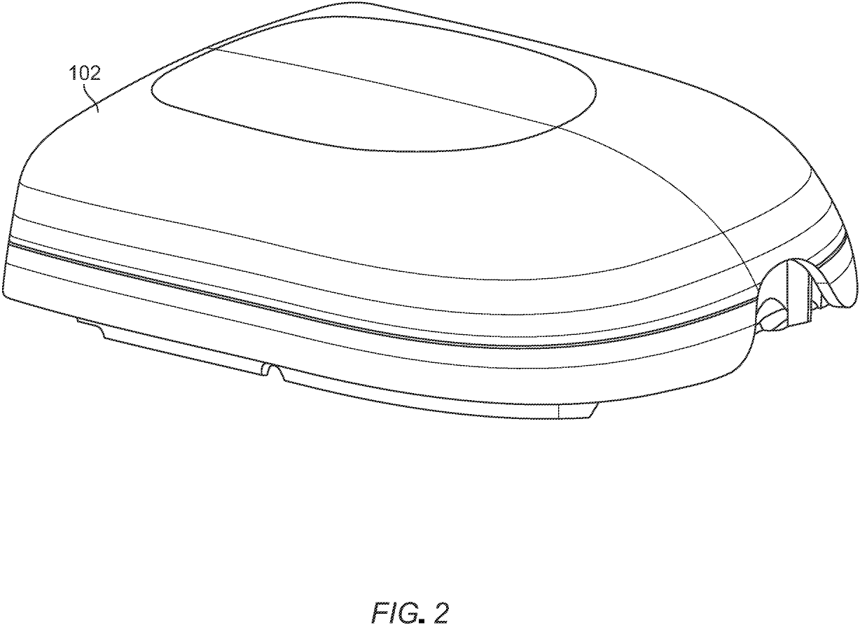

FIG. 1 illustrates a functional block diagram of an exemplary drug delivery system 100 suitable for implementing the systems and methods described herein. The drug delivery system 100 may implement (and/or provide functionality for) a medication delivery algorithm, such as an artificial pancreas (AP) application, to govern or control the automated delivery of a drug or medication, such as insulin, to a user (e.g., to maintain euglycemia - a normal level of glucose in the blood). The drug delivery system 100 may be an automated drug delivery system that may include a drug delivery device 102 (which may be wearable), an analyte sensor 108 (which may also be wearable), and a user device 105.

Drug delivery system 100, in an optional example, may also include an accessory device 106, such as a smartwatch, a personal assistant device, or the like, which may communicate with the other components of system 100 via either a wired or wireless communication links 191-193.

### User Device

The user device 105 may be a computing device such as a smartphone, a smartwatch, a tablet, a personal diabetes management (PDM) device, a dedicated diabetes therapy management device, or the like. In an example, user device 105 may include a processor 151, device memory 153, a user interface 158, and a communication interface 154. The user device 105 may also contain analog and/or digital circuitry that may be implemented as a processor 151 for executing processes based on programming code stored in device memory 153, such as user application 160 to manage a user’s blood glucose levels and for controlling the delivery of the drug, medication, or therapeutic agent to the user, as well for providing other functions, such as calculating carbohydrate-compensation dosage, a correction bolus dosage and the like as discussed below. The user device 105 may be used to activate, deactivate, trigger a needle/canula insertion, program, adjust settings, and/or control operation of drug delivery device 102 and/or the analyte sensor 103 as well as the optional smart accessory device 106.

The processor 151 may also be configured to execute programming code stored in device memory 153, such as the user app 160. The user app 160 may be a computer application that is operable to deliver a drug based on information received from the analyte sensor 103, the cloud-based services 111 and/or the user device 105 or optional accessory device 106. The memory 153 may also store programming code to, for example, operate the user interface 158 (e.g., a touchscreen device, a camera or the like), the communication interface 154 and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 is an AP application, the processor 151 is also configured to execute a diabetes treatment plan (which may be stored in a memory) that is managed by user app 160. In addition to the functions mentioned above, when user app 160 is an AP application, it may further provide functionality to determine a carbohydrate-compensation dosage, a correction bolus dosage and determine a real-time basal dosage according to a diabetes treatment plan. In addition, as an AP application, user app 160 provides functionality to output signals to the drug delivery device 102 via communications interface 154 to deliver the determined bolus and basal dosages.

The communication interface 154 may include one or more transceivers that operate according to one or more radio-frequency protocols. In one embodiment, the transceivers may comprise a cellular transceiver and a Bluetooth® transceiver. The communication interface 154 may be configured to receive and transmit signals containing information usable by user app 160.

User device 105 may be further provided with one or more output devices 155 which may be, for example, a speaker or a vibration transducer, to provide various signals to the user.

### Drug Delivery Device

In various exemplary embodiments, drug delivery device 102 may include a reservoir 124 and drive mechanism 125, which are controllable by controller 121, executing a medication delivery algorithm (MDA) 129 stored in memory 123, which may perform some or all of the functions of the AP application described above, such that user device 105 may be unnecessary for drug delivery device 102 to carry out drug delivery and control. Alternatively, controller 121 may act to control reservoir 124 and drive mechanism 125 based on signals received from user app 160 executing on a user device 105 and communicated to drug delivery device 102 via communication link 194. Drive mechanism 125 operates to longitudinally translate a plunger through the reservoir, so as to force the liquid drug through an outlet fluid port to needle / cannula 186.

In an alternate embodiment, drug delivery device 102 may also include an optional second reservoir 124-2 and second drive mechanism 125-2 which enables the independent delivery of two different liquid drugs. As an example, reservoir 124 may be filled with insulin, while reservoir 124-2 may be filled with Pramlintide or GLP-1. In some embodiments, each of reservoirs 124, 124-2 may be configured with a separate drive mechanism 125, 125-2, respectively, which may be separately controllable by controller 121 under the direction of MDA 129. Both reservoirs 124, 124-2 may be connected to a common needle / cannula 186.

Drug delivery device 102 may be optionally configured with a user interface 127 providing a means for receiving input from the user and a means for outputting information to the user. User interface 127 may include, for example, light-emitting diodes, buttons on a housing of drug delivery device 102, a sound transducer, a micro-display, a microphone, an accelerometer for detecting motions of the device or user gestures (e.g., tapping on a housing of the device) or any other type of interface device that is configured to allow a user to enter information and/or allow drug delivery device 102 to output information for presentation to the user (e.g., alarm signals or the like).

Drug delivery device 102 includes a patient interface 186 for interfacing with the user to deliver the liquid drug. Patient interface may be, for example, a needle or cannula for delivering the drug into the body of the user (which may be done subcutaneously, intraperitoneally, or intravenously). Drug delivery device 102 further includes a mechanism for inserting the needle / cannula 186 into the body of the user, which may be integral with or attachable to drug delivery device 102. The insertion mechanism may comprise, in one embodiment, an actuator that inserts the needle / cannula 186 under the skin of the user and thereafter retracts the needle, leaving the cannula in place. The actuator may be triggered by user device 105 or may be a manual firing mechanism comprising springs or other energy storing mechanism, that causes the needle / cannula 186 to penetrate the skin of the user.

In one embodiment, drug delivery device 102 includes a communication interface 126, which may be a transceiver that operates according to one or more radio-frequency protocols, such as Bluetooth®, Wi-Fi, near-field communication, cellular, or the like. The controller 121 may, for example, communicate with user device 105 and an analyte sensor 108 via the communication interface 126.

In some embodiments, drug delivery device 102 may be provided with one or more sensors 184. The sensors 184 may include one or more of a pressure sensor, a power sensor, or the like that are communicatively coupled to the controller 121 and provide various signals. For example, a pressure sensor may be configured to provide an indication of the fluid pressure detected in a fluid pathway between the patient interface 186 and reservoir 124. The pressure sensor may be coupled to or integral with the actuator for inserting the patient interface 186 into the user. In an example, the controller 121 may be operable to determine a rate of drug infusion based on the indication of the fluid pressure. The rate of drug infusion may be compared to an infusion rate threshold, and the comparison result may be usable in determining an amount of insulin onboard (IOB) or a total daily insulin (TDI) amount. In one embodiment, analyte sensor 108 may be integral with drug delivery device 102.

Drug delivery device 102 further includes a power source 128, such as a battery, a piezoelectric device, an energy harvesting device, or the like, for supplying electrical power to controller 121, memory 123, drive mechanisms 125 and/or other components of drug delivery device 102.

Drug delivery device 102 may be configured to perform and execute processes required to deliver doses of the medication to the user without input from the user device 105 or the optional accessory device 106. As explained in more detail, MDA 129 may be operable, for example, to determine an amount of insulin to be delivered, IOB, insulin remaining, and the like and to cause controller 121 to activate drive mechanism 125 to deliver the medication from reservoir 124. MDA 129 may take as input data received from the analyte sensor 108 or from user app 160.

The reservoirs 124, 124-2 may be configured to store drugs, medications or therapeutic agents suitable for automated delivery, such as insulin, Pramlintide, GLP-1, co-formulations of insulin and GLP-1, morphine, blood pressure medicines, chemotherapy drugs, fertility drugs or the like.

Drug delivery device 102 may be a wearable device and may be attached to the body of a user, such as a patient or diabetic, at an attachment location and may deliver any therapeutic agent, including any drug or medicine, such as insulin or the like, to a user at or around the attachment location. A surface of drug delivery device 102 may include an adhesive to facilitate attachment to the skin of a user.

When configured to communicate with an external device, such as the user device 105 or the analyte sensor 108, drug delivery device 102 may receive signals over the wired or wireless link 194 from the user device 105 or from the analyte sensor 108. The controller 121 of drug delivery device 102 may receive and process the signals from the respective external devices as well as implementing delivery of a drug to the user according to a diabetes treatment plan or other drug delivery regimen.

### Accessory Device

Optional accessory device 107 may be, a wearable smart device, for example, a smart watch (e.g., an Apple Watch®), smart eyeglasses, smart jewelry, a global positioning system-enabled wearable, a wearable fitness device, smart clothing, or the like. Similar to user device 105, the accessory device 107 may also be configured to perform various functions including controlling drug delivery device 102. For example, the accessory device 107 may include a communication interface 174, a processor 171, a user interface 178 and a memory 173. The user interface 178 may be a graphical user interface presented on a touchscreen display of the smart accessory device 107. The memory 173 may store programming code to operate different functions of the smart accessory device 107 as well as an instance of the user app 160, or a pared-down version of user app 160 with reduced functionality. In some instances, accessory device 107 may also include sensors of various types.

### Analyte Sensor

The analyte sensor 108 may include a controller 131, a memory 132, a sensing/measuring device 133, an optional user interface 137, a power source/energy harvesting circuitry 134, and a communication interface 135. The analyte sensor 108 may be communicatively coupled to the processor 151 of the management device 105 or controller 121 of drug delivery device 102. The memory 132 may be configured to store information and programming code 136.

The analyte sensor 108 may be configured to detect one or multiple different analytes, such as glucose, lactate, ketones, uric acid, sodium, potassium, alcohol levels or the like, and output results of the detections, such as measurement values or the like. The analyte sensor 108 may, in an exemplary embodiment, be configured as a continuous glucose monitor (CGM) to measure a blood glucose values at a predetermined time interval, such as every 5 minutes, every 1 minute, or the like. The communication interface 135 of analyte sensor 108 may have circuitry that operates as a transceiver for communicating the measured blood glucose values to the user device 105 over a wireless link 195 or with drug delivery device 102 over the wireless communication link 108. While referred to herein as an analyte sensor 108, the sensing/measuring device 133 of the analyte sensor 108 may include one or more additional sensing elements, such as a glucose measurement element, a heart rate monitor, a pressure sensor, or the like. The controller 131 may include discrete, specialized logic and/or components, an application-specific integrated circuit, a microcontroller or processor that executes software instructions, firmware, programming instructions stored in memory (such as memory 132), or any combination thereof.

Similar to the controller 121 of drug delivery device 102, the controller 131 of the analyte sensor 108 may be operable to perform many functions. For example, the controller 131 may be configured by programming code 136 to manage the collection and analysis of data detected by the sensing and measuring device 133.

Although the analyte sensor 108 is depicted in FIG. 1 as separate from drug delivery device 102, in various embodiments, the analyte sensor 108 and drug delivery device 102 may be incorporated into the same unit. That is, in various examples, the analyte sensor 108 may be a part of and integral with drug delivery device 102 and contained within the same housing as drug delivery device 102 or an attachable housing thereto. In such an example configuration, the controller 121 may be able to implement the functions required for the proper delivery of the medication alone without any external inputs from user device 105, the cloud-based services 111, another sensor (not shown), the optional accessory device 106, or the like.

### Cloud-Based Services

Drug delivery system 100 may communicate with or receive services from cloud-based services 111. Services provided by cloud-based services 111 may include data storage that stores personal or anonymized data, such as blood glucose measurement values, historical IOB or TDI, prior carbohydrate-compensation dosage, and other forms of data. In addition, the cloud-based services 111 may process anonymized data from multiple users to provide generalized information related to TDI, insulin sensitivity, IOB and the like. The communication link 115 that couples the cloud-based services 111 to the respective devices 102, 105, 106, 108 of system 100 may be a cellular link, a Wi-Fi link, a Bluetooth® link, or a combination thereof.

### Communication Links

The wireless communication links 115 and 191-196 may be any type of wireless link operating using known wireless communication standards or proprietary standards. As an example, the wireless communication links 191-196 may provide communication links based on Bluetooth®, Zigbee®, Wi-Fi, a near-field communication standard, a cellular standard, or any other wireless protocol via the respective communication interfaces 126, 135, 154 and 174.

### Operational Example

In an operational example, user application 160 implements a graphical user interface that is the primary interface with the user and is used to start and stop drug delivery device 102, program basal and bolus calculator settings for manual mode as well as program settings specific for automated mode (hybrid closed-loop or closed-loop).

User app 160, provides a graphical user interface 158 that allows for the use of large text, graphics, and on-screen instructions to prompt the user through the set-up processes and the use of system 100. It will also be used to program the user’s custom basal insulin delivery profile, check the status, of drug delivery device 102, initiate bolus doses of insulin, make changes to a patient’s insulin delivery profile, handle system alerts and alarms, and allow the user to switch between automated mode and manual mode.

User app 160 may be configured to operate in a manual mode in which user app 160 will deliver insulin at programmed basal rates and user-defined bolus amounts with the option to set temporary basal profiles. The controller 121 will also have the ability to function as a sensor-augmented pump in manual mode, using sensor glucose data provided by the analyte sensor 108 to populate the bolus calculator.

User app 160 may be configured to operate in an automated mode in which user app 160 supports the use of multiple target blood glucose values. For example, in one embodiment, target blood glucose values can range from 110-150 mg/dL, in 10 mg/dL increments, in 5 mg/dL increments, or other increments, but preferably 10 mg/dL increments. The experience for the user will reflect current setup flows whereby the healthcare provider assists the user to program basal rates, glucose targets and bolus calculator settings. These in turn will inform the user app 160 for insulin dosing parameters. The insulin dosing parameters will be adapted over time based on the total daily insulin (TDI) delivered during each use of drug delivery device 102. A temporary hypoglycemia protection mode may be implemented by the user for various time durations in automated mode. With hypoglycemia protection mode, the algorithm reduces insulin delivery and is intended for use over temporary durations when insulin sensitivity is expected to be higher, such as during exercise.

The user app 160 (or MDA 129) may provide periodic insulin micro-boluses based upon past glucose measurements and/or a predicted glucose over a prediction horizon (e.g., 60 minutes). Optimal post-prandial control may require the user to give meal boluses in the same manner as current pump therapy, but normal operation of the user app 160 will compensate for missed meal boluses and mitigate prolonged hyperglycemia. The user app 160 uses a control-to-target strategy that attempts to achieve and maintain a set target glucose value, thereby reducing the duration of prolonged hyperglycemia and hypoglycemia.

In some embodiments, user device 105 and the analyte sensor 108 may not communicate directly with one another. Instead, data (e.g., blood glucose readings) from analyte sensor may be communicated to drug delivery device 102 via link 196 and then relayed to user device 105 via link 194. In some embodiments, to enable communication between analyte sensor 108 and user device 105, the serial number of the analyte sensor must be entered into user app 160.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value and insulin on board (IOB). IOB is estimated by user app 160 taking into account any manual bolus and insulin delivered by the algorithm.

### Description of Embodiments

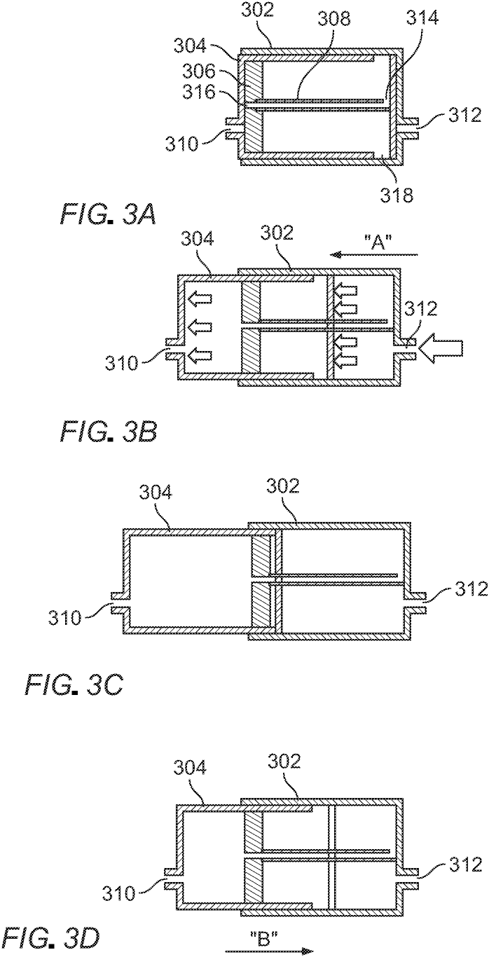

The primary embodiment of the invention is shown schematically in FIGS. 3(A-C). FIG. 3A shows the double reservoir, in an empty configuration in which inner reservoir 304 has been linearly translated the entire way into outer reservoir 302. Outer reservoir 302 is fixed with respect to a housing of drug delivery device 102 and inner reservoir 304 may be linearly translated into or out of outer reservoir 302 using external or internal drive mechanisms, several of which are discussed later herein. The static plunger 306 is fixed to outer reservoir 302 through hollow tube 308.

FIG. 3A shows the hollow tube 308 rigidly attached to a center portion of a back wall of outer reservoir 302. Hollow tube 308 defines fluid ports 314, 316 at either end thereof such that fluid in outer reservoir 302 may be forced through hollow tube 308 into inner reservoir 304. Static plunger 306 is disposed within inner reservoir 304 and acts to force fluid from inner reservoir 304 through fluid port 310 as the inner reservoir 304 is moved into outer reservoir 302.

Preferably, there is a fluid seal 318 defined around the circumference of an exterior surface of inner reservoir 304 so as to create a fluid seal between the exterior surface of inner reservoir 302 and an interior surface of outer reservoir 304, to prevent any fluid contained within outer reservoir 302 from leaking to the outside. Likewise, static plunger 306 is configured with a fluid seal around an outer circumference thereof to create a seal between plunger 306 and an interior wall of inner reservoir 304, to contain fluid in inner reservoir 304. Additional seals (not shown) may be provided between static plunger 306 and hollow tube 308 to prevent leakage between inner reservoir 304 and outer reservoir 302 and between hollow tube 308 and the end wall of inner reservoir 304, which acts as a plunger as inner reservoir 304 moves in direction “B” into outer reservoir 302.

FIG. 3B shows the process of filling the double reservoirs 302, 304. Adding fluid to the system through fluid port 312 defined in an end wall of outer reservoir 302 results in a pressure increase inside outer reservoir 302. Because hollow tube 308 fluidly couples outer reservoir 302 with inner reservoir 304, the fluid travels from outer reservoir 302 to inner reservoir 304 through the hollow tube 308, causing an increase in pressure inside of inner reservoir 304. Note that hollow tube 308 extends through static plunger 306 so as to allow fluid to enter the sealed end of inner reservoir 304. As the reservoirs continue to be filled, the built-up fluid pressure inside both reservoirs overcomes system frictions and starts to move inner reservoir 304 in direction “A”, and out of outer reservoir 302 until it reaches the fully filled configuration, shown in FIG. 3C, when the end wall of inner reservoir 304 contacts the static plunger 306. In alternative embodiments, the reservoirs 302, 304 may be separated before filling, in the position shown in FIG. 3C, and then filled when separated. In this case, the filling of the reservoirs does not force inner reservoir 304 in direction “A”. As the user inserts the liquid drug into either or both of reservoirs 302, 304, air is pushed out.

From the fully filled configuration shown in FIG. 3C, the fluid contained in both reservoirs may thereafter be dispensed. FIG. 3D shows the process of dispensing the fluid. Inner reservoir 304 is linearly translated into outer reservoir 302 in a controlled manner by an external driving force, which linearly translates inner reservoir 304 in direction “B” and into outer reservoir 302. Several example drive mechanisms providing the external driving force are disclosed herein. Forcing inner reservoir 304 into outer reservoir 302 reduces the available volume inside both reservoirs 302, 304 and pressurizes the contained fluid. When the fluid pressure exceeds the applied back pressure, fluid leaves the pump mechanism through outlet fluid port 310 defines on a wall of inner reservoir 304. The dispensing of fluid continues until both reservoirs reach the empty configuration, shown in FIG. 3A, when the end wall of inner reservoir 304 contacts the end wall of outer reservoir 302.

The positions of both fluid fill port 312 and fluid outlet port 310 are flexible so as to be able to be located on either of inner reservoir 304 or outer reservoir 302. The option which best fits the system requirements may be chosen for implementation. For example, having the fluid port on the moving, inner reservoir 304 may create challenges around integration of the pumping system with other subsystems of drug delivery device 102. In various embodiments, either of fluid ports 310, 312 may act as an inlet fluid port of an outlet fluid port. In such cases, it may be necessary to seal the fluid port acting as the outlet port during filling of the reservoirs and to seal the fluid port acting as the inlet port during dispending of the fluid. In some embodiments, the pumping mechanism may be configured with only one of fluid ports 310, 312, which acts as both an inlet fluid port and an outlet fluid port.

As explained above, an accurate dispensing of fluid requires an accurately controlled motion of inner reservoir 304 into outer reservoir 302. In some embodiments, a clutch may be provided between the drive mechanism and the reservoir to which the drive mechanism is connected. This allows the drive mechanism to be disengaged from the reservoir to allow the user to fill the reservoir (e.g., wherein the reservoir is required, during the filling process, to move in the opposite direction from its motion during the dispensing process). Once the filling is complete, the clutch may be engaged to connect the drive mechanism to the reservoir and the fluid may thereafter be dispensed through actuation of the drive mechanism. Several variations of the primary embodiment featuring different drive mechanisms will now be disclosed.

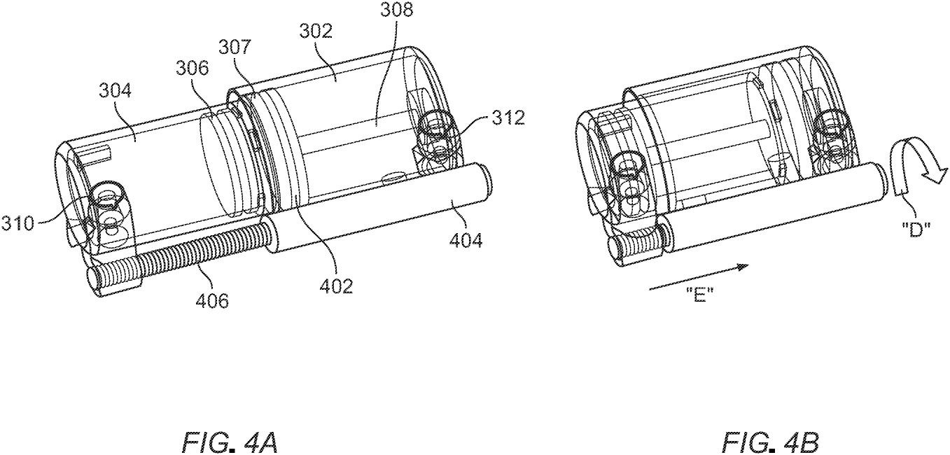

FIGS. 4(A-B) show a first extension of the primary embodiment in which the inner reservoir 304 is linearly translated into outer reservoir 302 using a leadscrew 406. Leadscrew 406 is attached to inner reservoir 304 as shown in FIG. 4A. Over molding, heat sticking, or adhesives can be used to ensure a secure bond between leadscrew 406 and inner reservoir 304. A tube nut 404, which is a slender tube having an interior threaded engagement with leadscrew 406, is engaged with the end of leadscrew 406 opposite the end which is engaged with inner reservoir 304. The pumping actuation occurs by rotating the tube nut 404 in direction “D”, as shown in FIG. 4B, which causes inner reservoir 304 to linearly translate in direction “E” into outer reservoir 302. Because the axial motion of tube nut 404 is restricted, the rotation of tube nut 404 results in an axial motion of leadscrew 406 and inner reservoir 304 via the threaded engagement between leadscrew 406 and tube nut 404.

FIGS. 4(A-B) also show fluid seal 402, which extends around an outside circumference of moving plunger 307 to create a fluid seal between the exterior surface of inner reservoir 304 and the interior surface of outer reservoir 302. Fluid seal 402 prevents fluid contained in outer reservoir 302 from escaping via the space between outer reservoir 302 and inner reservoir 304. FIGS. 4(A-B) show that plunger 306 is fitted with an O-ring around an outside circumference thereof to create a fluid seal between plunger 306 and an interior surface of inner reservoir 304, to prevent fluid contained in inner reservoir 304 from escaping. Additionally, a fluid seal (not shown) may be provided between hollow tube 308 and moving plunger 307 to prevent leakage between inner reservoir 304 and outer reservoir 302.

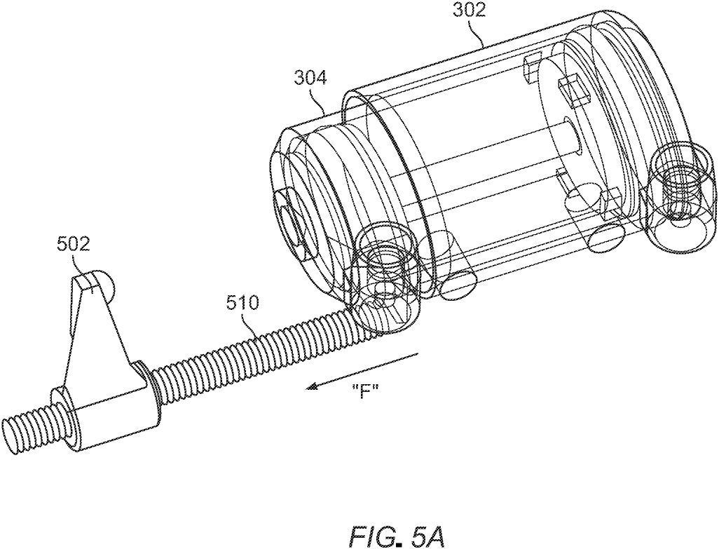

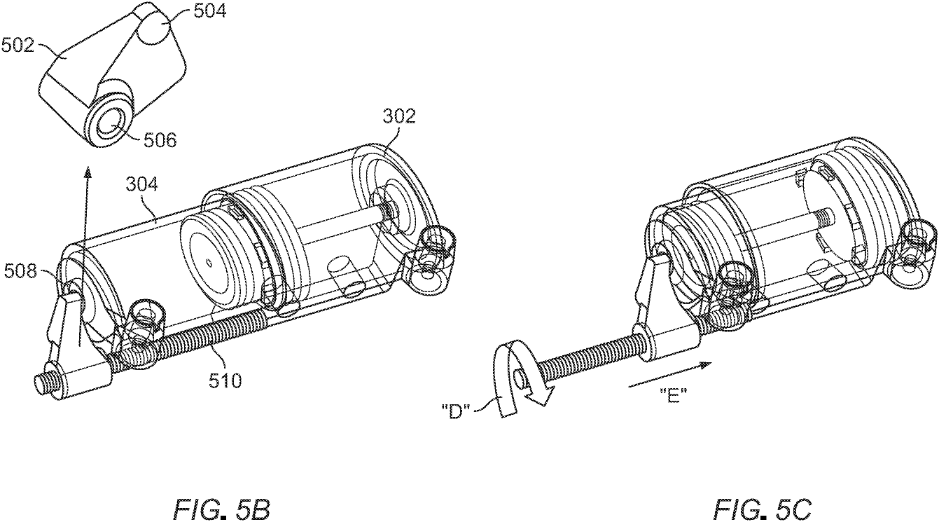

FIGS. 5(A-C) show a second extension of the primary embodiment in which leadscrew 510 is in threaded engagement with a pusher member 502 via a threaded through hole 506 defined in pusher member 502. Pusher member 502 moves axially along leadscrew 510 as leadscrew 510 rotates, but rotation of pusher element 502 about the axis of leadscrew 510 is restricted FIG. 5A, shows an initial state wherein neither reservoir is filled. Reservoir 304 is not in contact with pusher member 502 in the initial, unfilled state. As reservoirs 302, 304 are filled, inner reservoir 304 moves in direction “F”, eventually engaging with pusher member 502 as shown in FIG. 5B. It should be noted that reservoir 304 moves in direction “F” by virtue of the pressure created by the filling of reservoirs 302, 304, and not by action of leadscrew 510 or movement of pusher member 508. Once reservoirs 302, 304 are in a filled state, as shown in FIG. 5B, the action of leadscrew 510 will cause inner reservoir 304 to move in direction “E”, as shown in FIG. 5C.

Leadscrew 510 may be rotated via any known means. For example, the tube nut 404 shown in FIGS. 4(A-B) may also be used in this variation; alternatively or additionally, a motor may be used. The rotation of leadscrew 510 in direction “D” results in a linear translation of pusher member 502 along the longitudinal axis of leadscrew 510 in direction “E”. In some embodiments, pusher member 502 may be configured with a protruding feature 504 which engages a depression 508 defined on an exterior of the end wall of inner reservoir 304. In some embodiments, protruding feature 504 may be semi-spherical in shape and the depression 508 may be concave, or vice versa. In yet other embodiments, any other type of engagement between pusher member 502 and the exterior of the end wall of inner reservoir 304 may be used. As pusher member 502 is linearly translated in direction “E” by rotation of leadscrew 510 in direction “D”, pusher 502 engages the exterior end wall of inner reservoir 304 and moves inner reservoir 304 in direction “E” into outer reservoir 302. A significant advantage of this embodiment compared to the embodiment shown in FIGS. 4(A-B) is the absence of off-axis loading applied to inner reservoir 304. As such, inner reservoir 304 always remains coaxially aligned with outer reservoir 302.

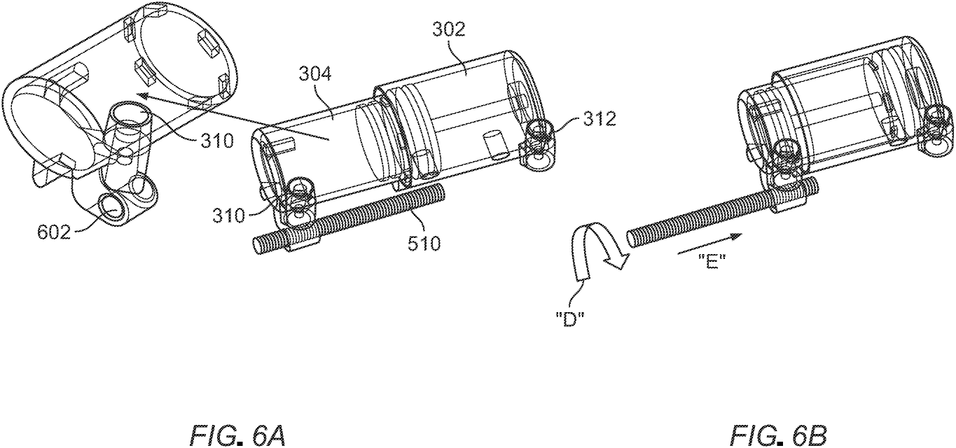

FIGS. 6(A-B) show a third extension of the primary embodiment in which the pusher member 502 of the extension shown in FIGS. 5(A-B) is integrated with the body of inner reservoir 304 as indicated by reference number 602. Note that, in the variation shown in FIG. 6A, pusher member 602 is also integrated with fluid port 310; however, as will be realized, pusher member 602 may be defined on inner reservoir 304 separate from fluid port 310. Pusher member 602 defines a through hole 604 therein containing interior threads. Pusher member 602 is in threaded engagement with leadscrew 510 such that rotation of leadscrew 510 in direction “D” results in a controlled linear translation of the inner reservoir 304 in direction “E”, into outer reservoir 302. As with previous embodiments, leadscrew 510 may be rotated via a tube nut of the type shown in FIGS. 4(A-B) as reference number 404, or by any other known means.

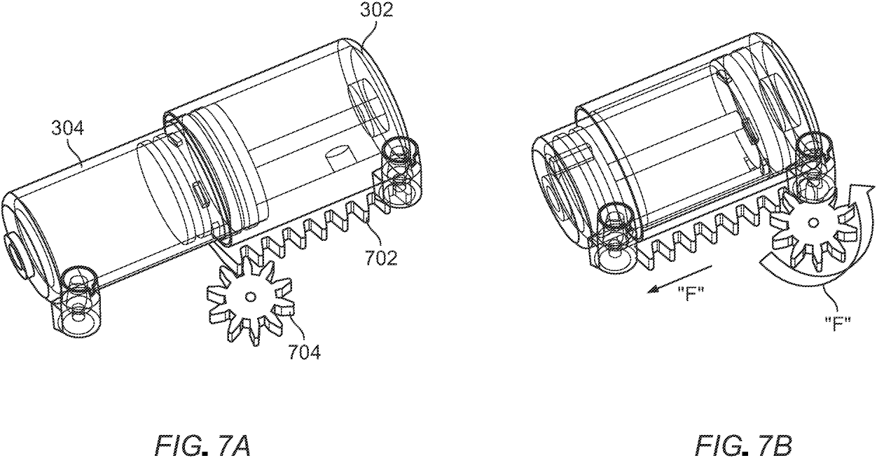

FIGS. 7(A-B) show a fourth extension of the primary embodiment. In this embodiment, inner reservoir 304 is preferably rigidly attached to the housing of the drug delivery device 102 and outer reservoir 302 is linearly translated so as to move over inner reservoir 304, thereby linearly translating inner reservoir 304 into outer reservoir 302. In this variation, the drive mechanism is a rack and pinion configuration. Outer reservoir 302 is configured with a rack gear 702 on an outside surface thereof. Pinion gear 704 is rotated by any known means in direction “F” to cause outer reservoir 302 to move in direction “F”, thereby linearly translating inner reservoir 304 into outer reservoir 302 by virtue of the linear motion of outer reservoir 302 in direction “F”.

Although the leadscrew / tube nut mechanism shown in the variations of FIGS. 4-6 provides an accurately controlled axial displacement of inner reservoir 304 and introduces mechanical advantage to the system, it can also reduce the energy efficiency of the system because of the additional friction caused by off-axis loading between the leadscrew thread and the tube nut threads, a limitation which is not present in this variation.

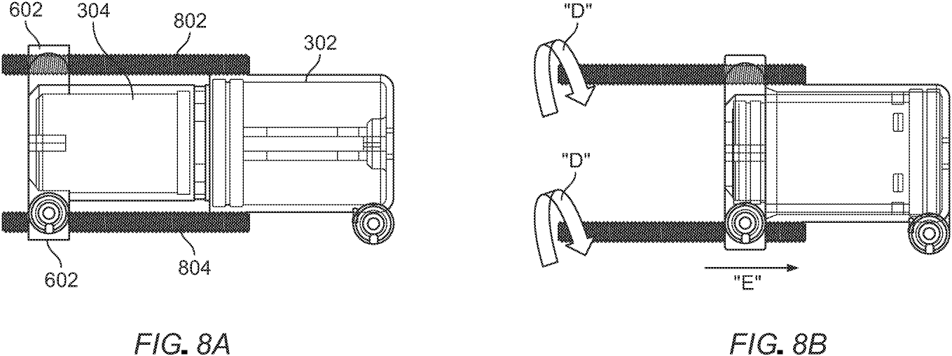

FIGS. 8(A-B) show a fifth extension of the primary embodiment. Leadscrews are not perfect mechanisms for off-axis loading as their frictional loss increases dramatically as off-axis loading is applied to the system. To minimize the effect of off-axis loading on the leadscrews, this variation uses two leadscrews 802, 804. Leadscrews 802, 804 are in threaded engagement with pusher members 602 of the type shown in FIGS. 6(A-B), which are integral with inner reservoir 304 and disposed on opposite sides of inner reservoir 304. A synchronized rotation of leadscrews 802, 804 in direction “D” therefore causes a linear translation of reservoir 304 in direction “E”, which drives inner reservoir 304 into outer reservoir 302. Leadscrews 802, 804 may be rotated using a tube nut of the type shown in FIGS. 4(A-B) as reference number 404, or any other known means. A variation (not shown) to using two leadscrews is to use one leadscrew on one side and a smooth cylindrical rod on the other, over which one of the pusher members 602 can slide. In this manner, only one leadscrew need be rotated while still overcoming the problem of off-axis loading.

The use of the dual leadscrews not only increases the energy efficiency of the system by minimizing or eliminating the off-axis moment applied to the leadscrew / tube nut interface but also improves the alignment between inner reservoir 304 and outer reservoir 302. The variation using two leadscrews (or one leadscrew and a smooth cylindrical rod) can be applied to any of the variations of the invention shown in FIGS. 4-6 discussed above. With respect to the embodiment shown in FIGS. 7(A-B), dual rack-and-pinion mechanisms could be used in the same manner as a dual leadscrew variation to achieve the same result.

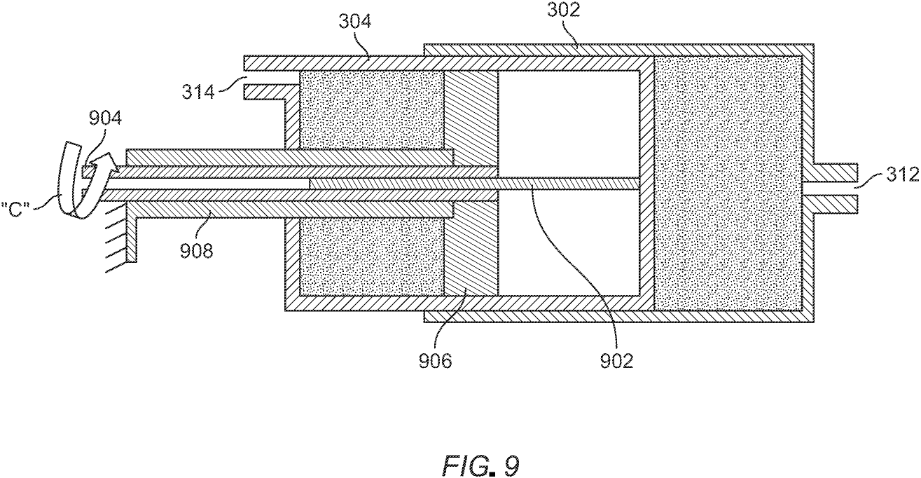

FIG. 9 shows, in schematic form, a secondary embodiment of the invention using the double reservoir configuration with central actuation. As discussed with respect to previous embodiments and variations, side actuation has disadvantages, such as creating misalignment between inner reservoir 304 and outer reservoir 302 and additional frictional loss at the leadscrew / tube nut interface. The embodiment shown in FIG. 9 overcomes these deficiencies by using a central actuation mechanism in which the leadscrew 902 is located inside of inner reservoir 304 and in contact with an end wall of inner reservoir 304. As such, leadscrew 902 is disposed coaxially with reservoirs 302, 304. In this case, the loading is aligned with the desired direction of motion of inner reservoir 304 and prevents misalignment between reservoirs 302, 304 and reduces the frictional loss in leadscrew 902.

FIG. 9 is a schematic representation of the double reservoir embodiment having central actuation, where leadscrew 902 with outer threads is in threaded engagement with tube nut 904 with inner threads. Plunger tube 908 is rigidly attached to a housing of the drug delivery device 102 and supports static plunger 906. In this embodiment, outer reservoir 302 is also rigidly attached to the housing of drug delivery device 102, and tube nut 904 is restricted from moving in the axial direction but is free to rotate about its longitudinal axis in direction “C”. Leadscrew 902 is moved axially by virtue of its threaded engagement with tube nut 904 to push inner reservoir 304 into outer reservoir 302 by virtue of the engagement between leadscrew 902 and the end wall of inner reservoir 304. In this embodiment, interfaces between inner reservoir 304 and outer reservoir 302, static plunger 906 and inner reservoir 304, and inner reservoir 304 and plunger tube 908 are fluidly sealed by means discussed previously with respect to other variations. Note that, in this embodiment, each of inner reservoir 304 and outer reservoir 302 must have individual fluid ports as the fluid is prevented from moving between inner reservoir 304 and outer reservoir 302. The coupling of the respective fluid ports from inner reservoir 304 and outer reservoir 302 occurs external to the pumping mechanism at any convenient location between the pumping mechanism and the patient interface.

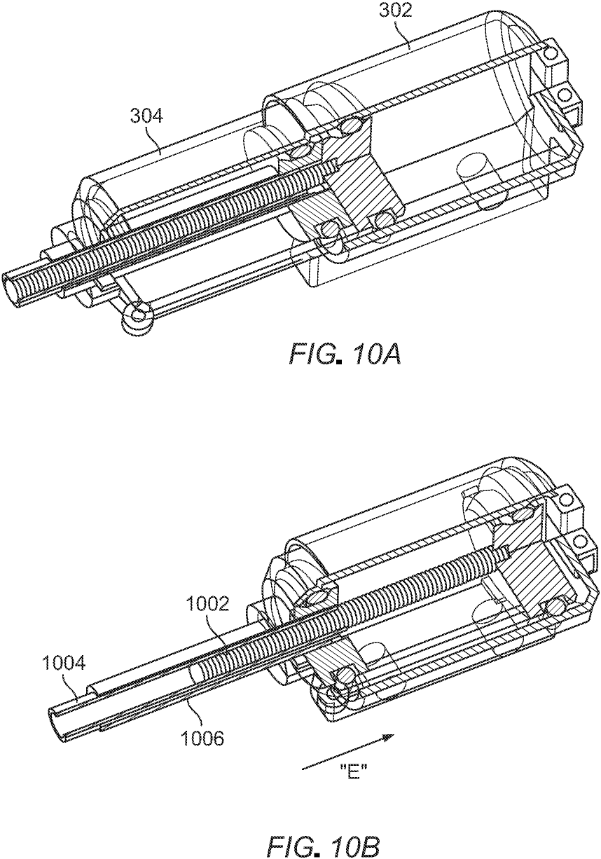

FIGS. 10(A-B) shows a cut-away view of this embodiment of the invention. Because of the threaded engagement between leadscrew 902 and tube nut 904, rotation of tube nut 904 moves leadscrew 902 in direction “E” to push on the end wall of inner reservoir 304 thereby moving inner reservoir 304 into outer reservoir 302.

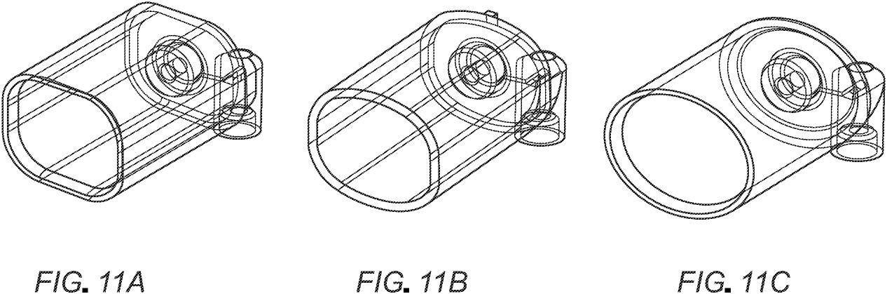

Exemplary embodiments of the invention disclosed herein have been presented with reservoirs having a circular cross-sectional shape. However, the invention is not intended to be limited thereto. In alternate embodiments, reservoirs having different cross-sectional shapes may be used, examples of which are shown in FIGS. 11(A-C). Although in the primary embodiment reservoirs may have a circular cross-sectional shape, other cross-sectional shapes such as rectangular with rounded edges (FIG. 11A), flattened circular (FIG. 11B), elliptical (FIG. 11C), etc. can be implemented without departing from the intended scope of the invention. While a rectangular cross-sectional shape may provide an increase in volume efficiency over a circular cross-sectional shape, the rectangular cross-sectional shape also has an increased risk of seal failure due to the sharper corners. Ideally, a combination of rectangular and elliptical shapes, as shown in FIGS. 11(A-C), can be a preferred option to achieve both reasonable sealing quality and volume efficiency.

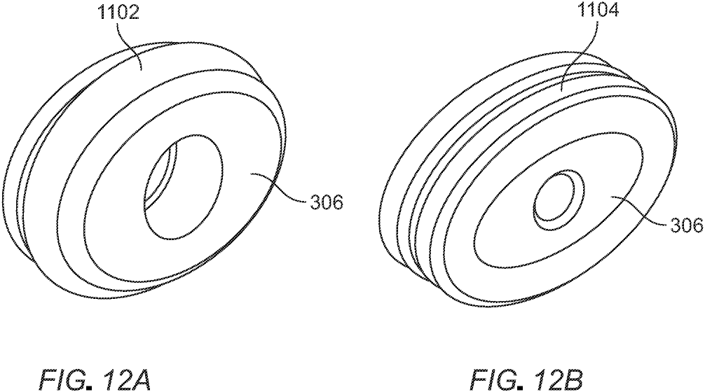

As described with respect to the embodiments and variations disclosed herein, some interfaces, such as the interface between inner reservoir 304 and outer reservoir 302 need to be sealed to achieve the expected performance of the pumping mechanism. In these cases, an O-ring may be used, as shown in FIG. 12A, which may be installed into grooves defined on the circumferential surface of plunger 306 and which will maintain the dynamic seal between plunger 306 and the interior surface of inner reservoir 304. Alternatively, a two-shot molding technique may be used to have the silicon rubber sealing element bonded to the plunger 306 as shown in FIG. 12B. The two-shot molding simplifies the assembly process.

The following examples pertain to various embodiments of the systems and methods disclosed herein for implementation of an automatic drug delivery system having a double reservoir pumping mechanism.

Example 1 is a first embodiment of a pumping mechanism comprising an outer reservoir, and inner reservoir, configured to linearly translate into and out of the other reservoir, static plunger disposed on the interior of the inner reservoir, a hollow tube supporting the static plunger fluidly coupling the inner reservoir and the other reservoir, one or more fluid ports and a drive mechanism for linearly translating the inner reservoir into the outer reservoir.

Example 2 is an extension of Example 1, or any other example disclosed herein, further comprising a fluid seal between the inner and outer reservoirs.

Example 3 is an extension of Example 1, or any other example disclosed herein, wherein the other reservoir is rigidly attached to a structure external to the pumping mechanism.

Example 4 is an extension of Example 3, or any other example disclosed herein, wherein the inner reservoir defines a through hole and the drive mechanism comprises a leadscrew engaged with the through hole such that a linear translation of the leadscrew causes a linear translation of the inner reservoir into the outer reservoir.

Example 5 is an extension of Example 4, or any other example disclosed herein, wherein the drive mechanism further comprises a tube nut in threaded engagement with the leadscrew such that a rotation of the tube nut causes the linear translation of the leadscrew.

Example 6 is an extension of Example 1, or any other example disclosed herein, wherein an exterior surface of the inner reservoir defines a depression, the drive mechanism comprising a pusher member defining a through hole having internal threads and a protrusion thereon, and a leadscrew in threaded engagement with the through hole of the pusher member, wherein a rotation of the leadscrew causes a linear translation of the pusher member to engage the protrusion with the depression on the inner reservoir two push the inner reservoir into the outer reservoir.

Example 7 is an extension of Example 6, or any other example disclosed herein, wherein the protrusion is semi-spherical in shape and the depression is concave.

Example 8 is an extension of Example 1, or any other example disclosed herein, wherein the drive mechanism comprises a pusher element integral with the inner reservoir and defining a through hole having internal threads and a leadscrew in threaded engagement with the through hole such that a rotation of leadscrew causes a linear translation of the pusher element and a linear translation of the inner reservoir into the outer reservoir.

Example 9 is an extension of Example 1, or any other example disclosed herein, wherein the drive mechanism comprises a rack gear disposed on the outer surface of the outer reservoir and a pinion gear engaged with the rack gear such that a rotation of the pinion gear causes a linear translation of the outer reservoir toward the end of reservoir.

Example 10 is an extension of Example 7, or any other example disclosed herein, wherein the inner reservoir defines a second through hole disposed on an exterior surface opposite the through hole wherein the drive mechanism further comprises a second leadscrew engaged with the second through hole such that a synchronized linear translation of the leadscrew and the second leadscrew causes a linear translation of the inner reservoir into the outer reservoir.

Example 11 is an extension of Example 8, or any other example disclosed herein, wherein the drive mechanism further comprises a second pusher element integral with the inner reservoir defining a through hole having internal threads and a second leadscrew in threaded engagement with the through hole of the second pusher element wherein a synchronized rotation of the leadscrews causes a linear translation of the inner reservoir into the outer reservoir.

Example 12 is an extension of Example 9, or any other example disclosed herein, wherein the drive mechanism further comprises a second rack gear disposed on an outer surface of the outer reservoir opposite the rack gear and a second pinion gear engaged with the second rack gear such that a synchronized rotation of the pinion gears causes a linear translation of the outer reservoir toward the inner reservoir.

Example 13 is an extension of Example 1, or any other example disclosed herein, wherein the inner reservoir and the outer reservoir have a cross-sectional shape selected from a group consisting of elliptical, flattened circular and rectangular with rounded corners.

Example 14 is an extension of Example 1, or any other example disclosed herein, further comprising a fluid seal disposed on a circumferential surface of the static plunger such as to create a fluid seal between the plunger and an interior surface of the inner reservoir.

Example 15 is an extension of Example 1, or any other example disclosed herein, further comprising a fluid seal between an interior surface of the outer reservoir and an exterior surface of the inner reservoir.

Example 16 is an extension of Example 1, or any other example disclosed herein, further comprising a fluid seal between the hollow tube in the static plunger.

Example 17 a second embodiment of a pumping mechanism comprising an outer reservoir, and inner reservoir configured to linearly translate into and out of the outer reservoir, a static plunger disposed on the interior of the inner reservoir, a leadscrew, disposed internal to the inner reservoir coaxial with the longitudinal axis of the inner reservoir and extending through the static plunger to contact an end wall of the inner reservoir and a tube nut, engaged with the leadscrew such that rotation of the tube nut causes a linear translation of the leadscrew pushing the inner reservoir into the outer reservoir.

Example 18 is an extension of Example 17, or any other example disclosed herein, wherein the outer reservoir is rigidly attached to a structure external to the pumping mechanism.

Example 19 is an extension of Example 18, or any other example disclosed herein, further comprising a hollow tube containing the tube nut, the hollow tube rigidly attached to a structure external to the pumping mechanism and supporting the static plunger.

Example 20 is extension of Example 19, or any other example disclosed herein, further comprising a drive mechanism for rotating the tube nut.

Example 21 is an extension of Example 20, or any other example disclosed herein, further comprising a first fluid port defined in a wall of the outer reservoir and a second fluid port defined in a wall of the inner reservoir.

Example 22 is an extension of Example 21, or any other example disclosed herein, wherein the first fluid port and second fluid port are fluidly coupled external to the pumping mechanism.

Example 23 is an extension of Example 17, or any other example disclosed herein, further comprising a fluid seal disposed on a circumferential surface of the static plunger such as to create a fluid seal between the plunger and an interior surface of the inner reservoir.

Example 24 is an extension of example 17, or any other example disclosed herein, further comprising a fluid seal between the inner reservoir and the outer reservoir.

Example 25 is an extension of example 17, or any other example disclosed herein, wherein the inner reservoir and the outer reservoir have a cross-sectional shape selected from a group consisting of elliptical, flattened circular and rectangular with rounded corners.

Example 26 is a third embodiment of a pumping mechanism comprising an outer reservoir, an inner reservoir configured to linearly translate into the outer reservoir and a drive mechanism for linearly translating the inner reservoir, wherein a portion of the inner reservoir acts as a plunger within the outer reservoir to force medication contained in the outer reservoir through a first fluid port.

Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

To those skilled in the art to which the invention relates, many modifications and adaptations of the invention may be realized. Implementations provided herein, including sizes, shapes, ratings compositions and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A pumping mechanism comprising:
an outer reservoir;
an inner reservoir, configured to linearly translate into the outer reservoir;
a static plunger disposed on the interior of the inner reservoir;
a hollow tube, supporting the static plunger and extending between the inner reservoir and the outer reservoir, thereby fluidly coupling the inner reservoir to the outer reservoir;
one or more fluid ports defined in either or both of the inner reservoir and the outer reservoir; and
a drive mechanism for linearly translating the inner reservoir into the outer reservoir.

2. The pumping mechanism of claim 1 further comprising:
a fluid seal between an interior surface of the outer reservoir and an exterior surface of the inner reservoir.

3. The pumping mechanism of claim 1 wherein the outer reservoir is rigidly attached to a structure external to the pumping mechanism.

4. The pumping mechanism of claim 1 wherein a portion attached to the inner reservoir defines a through hole, and the drive mechanism comprises:
a leadscrew engaged with the through hole, such that a linear translation of the leadscrew causes a linear translation of the inner reservoir into the outer reservoir.

5. The pumping mechanism of claim 4, wherein the drive mechanism further comprises:
a tube nut in threaded engagement with the leadscrew, such that a rotation of the tube nut causes the linear translation of the leadscrew.

6. The pumping mechanism of claim 5, wherein the tube nut is contained in the hollow tube, the hollow tube rigidly attached to a structure external to the pumping mechanism and supporting the static plunger.

7. The pumping mechanism of claim 1, wherein the one or more fluid ports comprise a first fluid port defined in a wall of the outer reservoir and a second fluid port defined in a wall of the inner reservoir.

8. The pumping mechanism of claim 7, wherein the first fluid port and second fluid port fluidly are coupled external to the pumping mechanism.

9. The pumping mechanism of claim 1, the drive mechanism comprising:
a pusher member defining a through hole having internal threads; and
a leadscrew in threaded engagement with the through hole of the pusher member;
wherein a rotation of the leadscrew causes a linear translation of the pusher member such that the pusher member pushes the inner reservoir into the outer reservoir.

10. The pumping mechanism of claim 9, wherein:
a mating element is defined on the pusher member; and
a corresponding mating element is defined on the exterior surface of the inner reservoir.

11. The pumping mechanism of claim 1 wherein the drive mechanism comprises:
a rack gear disposed on an outer surface of the outer reservoir; and
a pinion gear engaged with the rack gear;
wherein a rotation of the pinion gear causes a linear translation of the outer reservoir toward the inner reservoir, thereby causing the linear translation of the inner reservoir into the outer reservoir.

12. The pumping mechanism of claim 11 wherein the drive mechanism further comprises:
a second rack gear disposed on an outer surface of the outer reservoir opposite the rack gear; and
a second pinion gear engaged with the second rack gear;
wherein a synchronized rotation of the pinion gear and the second pinion gear causes a linear translation of the outer reservoir toward the inner reservoir, thereby causing the linear translation of the inner reservoir into the outer reservoir.

13. The pumping mechanism of claim 1, the drive mechanism comprising:
a pusher member integral with or attached to the inner reservoir, the pusher member defining a through hole having internal threads; and
a leadscrew in threaded engagement with the through hole of the pusher member;
wherein a rotation of the leadscrew causes a linear translation of the pusher element and a linear translation of the inner reservoir into the outer reservoir.

14. The pumping mechanism of claim 13, wherein the inner reservoir defines a second through hole disposed on the exterior surface of the inner reservoir opposite the through hole, the drive mechanism further comprising:
a second leadscrew engaged with the second through hole of the inner reservoir, such that a synchronized linear translation of the leadscrew and the second leadscrew causes a linear translation of the inner reservoir into the outer reservoir.

15. The pumping mechanism of claim 13, wherein the drive mechanism further comprises:
a second pusher member integral with or attached to the inner reservoir, the second pusher member defining a through hole; and
a cylindrical rod positioned in the through hole of the second pusher member.

16. The pumping mechanism of claim 1 wherein the inner reservoir and the outer reservoir have a cross-sectional shape selected from a group consisting of elliptical, flattened circular and rectangular with rounded corners.

17. The pumping mechanism of claim 1, further comprising:
a fluid seal disposed on a circumferential surface of the static plunger such as to create a fluid seal between the plunger and an interior surface of the inner reservoir.

18. The pumping mechanism of claim 1 further comprising:
a fluid seal between an interior surface of the outer reservoir and an exterior surface of the inner reservoir.

19. The pumping mechanism of claim 1 further comprising:
a fluid seal between the hollow tube and the static plunger.
